(19) United States
(12) Patent Application Publication
(10) Pub. No.: US 2018/0289595 A1
(43) Pub. Date: Oct. 11, 2018

(54) A Cosmetic Composition

(71) Applicant: Conopco, Inc., d/b/a UNILEVER, Englewood Cliffs, NJ (US)

(72) Inventors: Mingqi AO, Shanghai (CN); Axel Herve EKANI NKODO, Higher Bebington (GB); Naresh Dhirajlal GHATLIA, Bangalore (IN); Hangsheng LI, Shanghai (CN); Caigen YUAN, Shanghai (CN)

(73) Assignee: Conopco, Inc., d/b/a UNILEVER, Englewood Cliffs, NJ (US)

(21) Appl. No.: 15/766,880

(22) PCT Filed: Sep. 22, 2016
(86) PCT No.: PCT/EP2016/072606

§ 371 Date: Apr. 9, 2018

(30) Foreign Application Priority Data
Oct. 12, 2015 (CN) PCT/CN2015/091765
Nov. 9, 2015 (EP) 15193613.5

(51) Int. Cl. A61K 8/02; A61K 8/27; A61K 8/29; A61K 8/34; A61K 8/67; A61K 8/81; A61K 8/96; A61Q 19/02

(52) U.S. Cl. CPC A61K 8/0212; A61K 2800/43; A61K 8/0279; A61K 8/27; A61K 8/29; A61K 8/345; A61K 8/675; A61K 8/8117; A61K 8/8152; A61K 8/96; A61Q 19/02; A61K 2800/26; A61K 2800/412; A61K 2800/413; A61K 8/025

(57) ABSTRACT

The invention relates to a skin lightening composition, particularly to a composition in the form of a mask that can be applied to the desired topical surface for providing instant lightening benefits without the disadvantages of unnatural whiteness. This is effective due to the inclusion of high amounts of whitening particle along with select film forming polymer while ensuring that the amount of humectants is below a certain value.

### FIELD OF THE INVENTION

The invention relates to a skin lightening composition. The invention more particularly relates to a skin lightening composition in the form of a mask that can be applied to the face or any other topical surface of the body for providing instant lightening benefits without the disadvantages of unnatural whiteness that is often associated with such instant products.

### BACKGROUND OF THE INVENTION

Skin lightening is one of the most sought after cosmetic benefits people have been looking for in recent times. The exposure of the skin to the sun and other environmental irritants causes it to darken, often in uneven and blochy manner. Thus people, not only those living near the tropics who are naturally born with darker skin, but also those who live far from the tropics suffer from such cosmetically unpleasant appearance. There have been many cosmetic approaches used to tackle this problem.

One approach is to use foundations and make-up compositions (also known as colour cosmetics) which are high in coloured dyes or pigments which are applied to the face to mask the unevenness of skin appearance. This approach, although it gives instant change in the skin appearance, are often used by people in the media and entertainment industry whose images are captured on camera. For day to day use, people prefer a natural appearance since they are reluctant to appear “heavily made up”. Another approach for skin lightening and evenness of skin appearance is to use skin lightening agents which act through biological means to regulate the melanin on the skin thus providing a more lightened appearance. But such biological actives like niacinamide, though effective, take days, if not weeks to render the effectiveness.

Sunscreen compositions comprising organic (UVA and UVB) and/or inorganic sunscreen agents are also widely used. They protect the skin against the natural darkening of the skin when exposed to solar radiation by absorbing and then emitting the UV radiation or by blocking them completely. Inorganic sunscreens like zinc oxide and titanium dioxide are also widely used. These inorganic actives come in various particle sizes. The low particle size (of the order of ten of nanometers) act as sunscreen agents while those in the hundreds of nanometer particle size range are called pigments and offer instant whitening benefits. Unfortunately if higher amounts (higher than say 1% in the composition) is incorporated, they have the disadvantage of un-natural whitening of the skin which is not like by consumers.

The present inventors while being aware of the drawbacks of the above cosmetic technologies took it upon themselves the object of solving the problem to provide for a skin lightening composition which can give instant whitening benefits through incorporation of high amounts of inorganic whitening particles while at the same time giving the skin a natural appearance.

There is another class of cosmetic products known as face mask or clay mask or face packs. Such products consist of a cream or a mud pack or a paper based mask which is applied to the face for a short or long period of time (say 15 minutes to several hours e.g. overnight application). After the product is applied for the desired period of time, it is either wiped or rinsed off with water. Such products have been claimed to give benefits against problems like acne by removing the dead skin cells on the surface and by removing unwanted oil and other irritants. Such products have also been claimed to moisturize the skin through use of high levels of humectants and other moisturizing agents. U.S. Pat. No. 5,747,022 (Unilever, 1988) discloses one such face mask composition comprising a judicious mixture of different type of film forming polymers to achieve fast drying.

Since conventional types of compositions like sunscreens, creams and gels comprising conventional skin lightening agents were not found to give the above benefits, the present inventors explored the as yet unused vehicle of a face mask to study if it can be used to deliver instant lightening benefits. After extensive experimentation they found that it is possible to combine specific film forming polymers with high amounts of lightening inorganic particles and such a composition when applied on the skin is capable of providing the instant whitening benefits. Surprisingly, the benefit is seen to be enhanced not only by way of absolute skin lightening scores but the skin was seen to appear more natural when the composition is formulated by limiting the amount of humectants to be below a certain value. Many cosmetic compositions comprising polymers, whitening particles and humectants at varying concentrations have been disclosed e.g. in WO15039826 and WO14203913, but they do not disclose the inventive combination of the present invention i.e. specific film forming polymers, the whitening particles in combination with the need for the humectant concentration to be below a certain value.

It is thus an object of the present invention to provide for a composition that delivers instant whitening benefits through use of high concentration of inorganic whitening particles, while ensuring the retention of even skin appearance in its natural form.

### SUMMARY OF THE INVENTION

According to the first aspect of the present invention there is provided a cosmetic composition comprising
(a) 0.5 to 10% by weight of a film forming polymer selected from an acrylate polymer, a methacrylate polymer, a urethane polymer or co-polymers thereof;
(b) 1 to 25% by weight of a whitening particle;
   wherein the composition comprises less than 15% humectants.
According to another aspect of the present invention there is provided a method of lightening skin comprising the steps of
(i) applying the composition of the first aspect on to the preferred external skin surface for at least one minute; and
(ii) removing the composition from said surface by wiping the surface with a suitable wipe or rinsing the surface with water.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

By “A Cosmetic Composition” as used herein, is meant to include a composition for topical application to the skin of mammals, especially humans. Such a composition is preferably of the mask type. By a mask type is meant a composition that is applied to the desired skin surface and left on for a period of time (say from one minute to 12 hours) after which it is wiped or rinsed off with water. The composition may also be formulated into a product which is applied to a human body for improving the appearance, cleansing, odor control or general aesthetics. The composition of the present invention can be in the form of a liquid, lotion, cream, foam, scrub, or gel. It may be applied using ones fingers or applied with an implement. Non-limiting examples of such compositions include leave-on skin lotions or creams. “Skin” as used herein is meant to include skin on the face and body (e.g., neck, chest, back, arms, underarms, hands, legs, buttocks and scalp) and especially to the sun exposed parts thereof e.g the face, neck or even parts that are known to darken without sun exposure like the underarm.

The invention provides for a cosmetic composition comprising a film forming polymer selected from an acrylate polymer, a methacrylate polymer, a urethane polymer or co-polymers thereof; and a whitening particle; wherein the composition comprises less than 15% humectants.

“Film-forming polymer” as used herein refers to a polymer which is capable of forming a cohesive and continuous covering over the hair and/or skin when applied to the surface. Contact angle, as used herein, means the angle at which a water/vapor interface meets a solid surface at a temperature of 25° C. Such an angle may be measured with a goniometer or other water droplet shape analysis systems with water droplet of 5 μl and at 25° C. The requirement for film-forming polymer, as per the present invention is that the film-forming polymer is suitable to be employed in cosmetic composition. The film-forming polymer, for use in the present invention, preferably has a contact angle of at least 85°.

Not wishing to be bound by theory, the inventors believe that such a compact and continuous film covers on the skin surface and helps to reduce the loss of the whitening particles during washing or later through abrasion. The film forming polymer is generally water insoluble and is distinct from water soluble polymers like cross-linked polyacrylic acid polymers sold as Carbopol which are commonly used in cosmetic product as thickeners. By a water insoluble polymer is meant that the solubility of the polymer in water at 25° C. is less than 1 wt %, preferably less than 0.5 wt %, more preferably less than 0.1 wt % and most preferably less then 0.01 wt %.

The film forming polymer as per the invention is selected from an acrylate polymer, a methacrylate polymer, a urethane polymer or co-polymers thereof. Especially preferred are acrylate co-polymer and a urethane-acrylic co-polymer. Commercially available film forming polymers which may be used in the present invention are KOBO-50N available from Kobo and Hybridur 875 from Air Products. The film forming polymer is preferably included in 0.5-8%, more preferably in 1-6%, further more preferably 2-6% by weight of the composition.

A whitening particle is included in the cosmetic composition of the invention. The whitening particle is selected from one of a polymer bead or an inorganic particle with a refractive index higher than 1.8. When a polymer bead is included, it is preferably a hollow sphere. Suitable hollow spheres are those made of a styrene/acrylate co-polymer, polyurethane, or polyethylene. Polymer beads which may be included in the composition of the invention preferably have a particle size in the range of 80 to 700 nm. Commercially available hollow sphere polymer beads which may be used in the composition of the invention are Sunspheres™ (from Dow Chemicals). Alternatively and preferably the whitening particles are inorganic particles with a refractive index higher than 1.8, preferably higher than 2.0, more preferably higher than 2.5. Preferred inorganic particles as per the invention are titanium dioxide, zinc oxide, boron nitride or mixtures thereof. Most preferred inorganic particle is titanium dioxide. The inorganic particles for inclusion in the composition of the invention preferably have a mean particle size from 15 nm to 1 micron, preferably 80 to 400 nm. Commercially available titanium dioxide are available as Korons1171 from Korons and KowetTiO 203975 from Sensient.

The present inventors have determined that an important aspect that contributes to the benefits of the present invention is that humectants are present in less than 15%, preferably less than 13%, more preferably less than 9%, further more preferably less than 6% by weight of the composition. By humectants is meant a compound that is hygroscopic in nature and is generally used in cosmetic composition to provide moisturisation benefits to the substrate it is applied on. Humectant substances generally have several hydrophilic groups e.g. hydroxyl groups.

The most commonly used humectants in the cosmetic industry are the polyhydric alcohols e.g. alkylene glycols like propylene glycol, hexylene glycol or butylene glycol; and sugar alcohols like glycerol, sorbitol, xylitol or malitol. It is preferred that polyhydric alcohols are present in less than 15% by weight of the composition. An especially preferred polyhydric alcohol is glycerol. The composition as per the invention preferably comprises less than 3%, preferably less then 2%, further more preferably less than 1% by weight of a humectant. In an especially preferred composition, humectants are absent from the composition.

The composition of the invention may additionally comprise a surfactant. A surfactant of the anionic or non-ionic form is especially preferred. Suitable surfactants include fatty alcohols and esters of fatty acids. Especially preferred and widely and inexpensively available surfactants of the above classes which may be included in the compositions of the present invention are cetearyl alcohol, glyceryl monostearate and PEG-20 stearate. Such surfactants are preferably included in 1 to 20%, more preferably included in 2-10% by weight of the composition.

In addition to the whitening particle, the composition of the invention may comprise additional particulate matter. Suitable particles may be selected from one or more of chalk, talc, silica, and clays like kaolins and/or bentonite. The additional particulate material is preferably included in 1 to 30%, more preferably 5 to 20% by weight of the composition.

The composition preferably additionally comprises water. Water may be present in 20 to 96%, preferably 40 to 90%, more preferably 50 to 80% by weight of the composition.

The composition of the present invention can comprise a wide range of other optional components. The CTFA Personal care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, opacifying agents, conditioners, exfoliating agents, pH adjusters, preservatives, natural extracts, essential oils, skin sensates, skin soothing agents, and skin healing agents.

According to another aspect of the present invention there is provided a method of lightening skin comprising the steps of applying the composition of the invention on to the preferred external skin surface for at least one minute, preferably at least 10 minutes, and further more preferably at least 15 minutes. In certain aspects the product is left applied on the skin for up to 20 minutes, sometimes upto 30 minutes and in certain cases up to one hour. The second step of the method comprises removing the composition from the surface by wiping the surface with a suitable wipe or rinsing the surface with water. The method is preferably non-therapeutic. The composition is preferably left on to the desired skin surface from 1 to 60 minutes after which it may be removed. After the composition is applied on the skin for the above mentioned period of time, it may be removed from the surface by wiping off or rinsing off with water. Preferably the composition is rinsed off with water.

The invention will now be illustrated with the help of the following non-limiting examples.

### EXAMPLES

### Examples 1-4: Effect of Inclusion of Film Forming Polymer

TABLE 1

| | Example | | | |
|---|---|---|---|---|
| | 1, | Example 2, | Example 3, | Example 4, |
| Ingredient | wt % | wt % | wt % | wt % |
| Cetearyl alcohol | 1.6 | 1.6 | 1.6 | 1.6 |
| GMS | 2.0 | 2.0 | 2.0 | 2.0 |
| PEG-20 Stearate | 2.2 | 2.2 | 2.2 | 2.2 |
| Glycerine | 2.5 | 2.5 | 2.5 | 2.5 |
| Propylene glycol | 8.0 | 8.0 | 8.0 | 8.0 |
| Titanium dioxide | 15.0 | 15.0 | 15.0 | 15.0 |
| (Korons 260 nm) | | | | |
| Kobo 50N | — | 4.0 | 6.0 | 8.0 |
| Particulates | 12.25 | 12.25 | 12.25 | 12.25 |
| Xanthan gum | 0.4 | 0.4 | 0.4 | 0.4 |
| Water | To 100 | To 100 | To 100 | To 100 |
| ΔL after wash | 0.59 | 0.71 | 0.86 | 0.89 |

GMS: Glyceryl mono stearate which was purchased form FACT Asia Pacific Pte Ltd. Kobo 50N: is a formulation containing 48.5 to 51.5% film forming polymer of acrylate type, 1-3 to 1.7% laureth-21 non-ionic surfactant in water.

Particulates: were included at 12.25% in the composition (and comprised 1.75% silica (CAB-O-SIL M-5), 1.0% bentonite (NF BC) and 9.5% Kaolin (white)

The above compositions were tested for their skin lightening ability by carrying out the following test:

150 mg of the compostions were spread evenly on a 50#Bio-skin plate (50#BSP) in an area with 3 cm diameter circle. After allowing the sample to dry for 30 minutes, the BSP was dipped into water for 30 seconds, and then taken out to wash using a brushing machine (M235 Martindale, SDL ATLAS) with counter 20 and speed 59.4 rpm. After that, the BSP was rinsed using tap water for 20 seconds and the L* value was measured after it was completely dry. ΔL was obtained from (Lₛₐₘₚₗₑ−L_{blank}). Lₛₐₘₚₗₑ is the L values of 50#BSP with the composition after the above wash protocol while L_{blank} is the L value of a blank 50#BSP.

The data is summarized in Table-1 as the ΔL value. The data in Table-1 indicates that compositions as per the invention (Examples 2-4, where a film forming polymer was included at different concentrations) was able to provide superior skin lightening values as compared to a composition outside the invention (Example-1).

### Examples 5-7: Effect of Amount of Humectant

TABLE 2

| | Example 5, | Example 6, | Example 7, |
|---|---|---|---|
| Ingredient | wt % | wt % | wt % |
| Cetearyl alcohol | 1.6 | 1.6 | 1.6 |
| GMS | 2.0 | 2.0 | 2.0 |
| PEG-20 Stearate | 2.2 | 2.2 | 2.2 |
| Glycerine | 1.25 | 0.625 | 0.0 |
| Propylene glycol | 4.0 | 2.0 | 0.0 |
| Titanium dioxide | 15.0 | 15.0 | 15.0 |
| (Korons 260 nm) | | | |
| Kobo 50N | 2.0 | 2.0 | 2.0 |
| Particulates | 12.25 | 12.25 | 12.25 |
| Xanthan gum | 0.4 | 0.4 | 0.4 |
| Water | To 100 | To 100 | To 100 |
| ΔL after wash | 2.86 | 4.56 | 8.38 |

Particulates: were included at 12.25% in the composition as in Table-1.

The above compositions were tested for their skin lightening ability by carrying out the abovementioned test.

The data is summarized in Table-2 as the ΔL value. The data in Table-2 indicates that superior skin lightening values were obtained when the total amount of humectants was reduced.

### Example 8 to 10: Effect of Amount of Humectants at Different Amounts of Whitening Particle and Film Forming Polymer

Samples as shown in Table-3 were prepared and the ΔL values measured in a similar fashion, which is summarized in Table-3.

TABLE 3

| | | Example | Example |
|---|---|---|---|
| | Example 8, | 9, | 10, |
| Ingredient | wt % | wt % | wt % |
| Cetearyl alcohol | 1.6 | 1.6 | 1.6 |
| GMS | 2.0 | 2.0 | 2.0 |
| PEG-20 Stearate | 2.2 | 2.2 | 2.2 |
| Glycerine | 1.25 | 0.625 | 0.0 |
| Propylene glycol | 4.0 | 2.0 | 0.0 |
| Titanium dioxide | 7.6 | 7.6 | 7.6 |
| (Korons 260 nm) | | | |
| Kobo 50N | 4.0 | 4.0 | 4.0 |
| Particulates | 12.25 | 12.25 | 12.25 |
| Xanthan gum | 0.4 | 0.4 | 0.4 |
| Water | To 100 | To 100 | To 100 |
| ΔL after wash | 2.67 | 4.61 | 6.61 |

The data in Table-3 indicates similar trend as seen in Table-2.

### Examples 11-13: Effect of Type of Film Forming Polymer

Various compositions as shown in Table-4 were prepared using different types of film forming polymers and the data of ΔL values is summarized therein.

TABLE 4

| | Example 11, | Example 12, | Example 13, |
|---|---|---|---|
| Ingredient | wt % | wt % | wt % |
| Cetearyl alcohol | 1.6 | 1.6 | 1.6 |
| GMS | 2.0 | 2.0 | 2.0 |
| PEG-20 Stearate | 2.2 | 2.2 | 2.2 |
| Titanium dioxide | 7.6 | 7.6 | 7.6 |
| (Korons 260 nm) | | | |
| Kobo 50N | 0.0 | 4.0 | 0.0 |
| Hybridur 875 | 0.0 | 0.0 | 4.0 |
| Particulates | 12.25 | 12.25 | 12.25 |
| Xanthan gum | 0.4 | 0.4 | 0.4 |
| Water | To 100 | To 100 | To 100 |
| ΔL after wash | 2.50 | 6.61 | 4.88 |

Hybridur 875 is a polyurethane-2 and polymethyl methacrylate copolymer purchased from Air Products

The data in Table-4 indicates that compositions with different types of film forming polymers (Examples 12 and 13, gave better skin lightening efficacy as compared to a composition without any polymer (Example 11).

### Examples 14 to 16: Effect of Inclusion of Additional Emollients

Compositions as shown in Table-5 were prepared and the skin lightening efficacy (ΔL values) is summarized therein.

TABLE 5

| | | Example | | |
|---|---|---|---|---|
| | Example 10, | 14, | Example 15, | Example 16, |
| Ingredient | wt % | wt % | wt % | wt % |
| Cetearyl alcohol | 1.6 | 1.6 | 1.6 | 1.6 |
| GMS | 2.0 | 2.0 | 2.0 | 2.0 |
| PEG-20 Stearate | 2.2 | 2.2 | 2.2 | 2.2 |
| Titanium dioxide | 7.6 | 7.6 | 7.6 | 7.6 |
| (Korons 260 nm) | | | | |
| Kobo 50N | 4.0 | 4.0 | 4.0 | 4.0 |
| Emollient type | — | Stearic | Ethyl hexyl | Dimethicone |
| | | acid | salicylate | DC200 |
| Emollient, wt % | — | 2.5 | 2.5 | 2.5 |
| Particulates | 12.25 | 12.25 | 12.25 | 12.25 |
| Xanthan gum | 0.4 | 0.4 | 0.4 | 0.4 |
| Water | To 100 | To 100 | To 100 | To 100 |
| ΔL after wash | 6.61 | 8.12 | 8.37 | 10.41 |

The data in Table-5 indicates that in compositions as per the invention, the skin lightening efficacy is further enhanced when an emollient is additionally included (Examples 14 to 16).

1. A cosmetic composition comprising:
(a) 0.5 to 10% by weight of a film forming polymer selected from an acrylate polymer, a methacrylate polymer, a urethane polymer or co-polymers thereof where the film forming polymer has a contact angle of at least 85° which means the angle at which a water/vapor interface meets a solid surface at temperature of 25° C.;
(b) 1 to 25% by weight of a whitening particle;
wherein the composition comprises less than 6% humectants, wherein said composition is a face mask.

2. The composition as claimed in claim 1 wherein said whitening particle is selected from a polymer bead or an inorganic particle with a refractive index higher than 1.8.

3. The composition as claimed in claim 2 wherein said whitening particle is a hollow sphere made of a styrene/acrylate co-polymer, titanium dioxide, zinc oxide, boron nitride or mixtures thereof.

4. The composition as claimed in claim 3 wherein said particle is titanium dioxide.

5. The composition as claimed in claim 4 wherein the titanium dioxide has a particle size from 15 nm to 1 micron.

6. The composition as claimed in claim 1 wherein the humectant is a polyhydric alcohol.

7. The composition as claimed in claim 1 additionally comprising 1 to 20% by weight of a surfactant.

8. The composition as claimed in claim 1 additionally comprising 20 to 96% by weight water.

9. A method of lightening skin comprising the steps of:
(i) applying the composition as claimed in claim 1 on to the preferred external skin surface for at least one minute;
(ii) removing the composition from said surface by wiping the surface with a suitable wipe or rinsing the surface with water.

10. (canceled)

11. (canceled)

12. The composition as claimed in claim 1 wherein the composition further comprises niacinamide.

13. The composition as claimed in claim 1 wherein the composition further comprises sunscreen.

14. The composition as claimed in claim 1 wherein the composition further comprises natural extracts.
